(19) United States
(12) Patent Application Publication
(10) Pub. No.: US 2010/0227884 A1
(43) Pub. Date: Sep. 9, 2010

(54) AMIDE COMPOUNDS AND PLANT DISEASE CONTROLLING METHOD USING SAME

(75) Inventors: Mayumi Kubota, Toyonaka-shi (JP); Takashi Komori, Nerima-ku (JP)

Correspondence Address: SUGHRUE MION, PLLC, 2100 PENNSYLVANIA AVENUE, N.W., SUITE 800, WASHINGTON, DC, 20037, US

(73) Assignee: Sumitomo Chemical Company, Limited, Chuo-ku, Tokyo (JP)

(21) Appl. No.: 12/682,870

(22) PCT Filed: Oct. 7, 2008
(86) PCT No.: PCT/JP2008/068520

§ 371 Date: Apr. 13, 2010

(30) Foreign Application Priority Data
Oct. 15, 2007 (JP) 2007-267567

(51) Int. Cl. A01N 43/42; C07D 215/38; C07C 215/74; A01P 15/00

(52) U.S. Cl. 514/311; 546/169; 564/389

(57) ABSTRACT

The object of the present invention is to provide a compound having excellent controlling effect on plant diseases. An amide compound represented by the following formula (I) has excellent controlling effect on plant diseases:

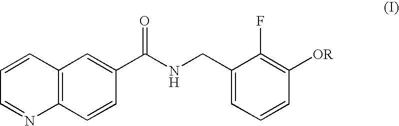

(in the formula, R denotes a C1-C5 straight-chain alkyl group).

### TECHNICAL FIELD

The present invention relates to amide compounds and a method for controlling plant diseases using the amide compounds.

### BACKGROUND ART

Hitherto, many compounds have been developed and put to practical use as active ingredients of plant disease controlling agents. However, these compounds do not always have sufficient controlling effect.

Patent Document 1: WO2005/033079

### DISCLOSURE OF INVENTION

### Problem to be Solved by the Invention

The object of the present invention is to provide a compound having excellent control effect for plant diseases.

### Means for Solving the Problem

As a result of intensive research conducted by the inventors in an attempt to find compounds having excellent control effect for plant diseases, it has been found that amide compounds represented by the following formula (I) have excellent control effect for plant diseases. Thus, the present invention has been accomplished.

The present invention provides an amide compound represented by the following formula (I):

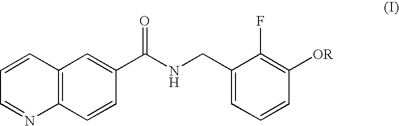

(in the formula, R represents a C1-C5 straight-chain alkyl group) (hereinafter referred to as “invented compound”), a plant disease controlling agent containing the invented compound as an active ingredient (hereinafter referred to as “invented controlling agent”), and a method for controlling plant diseases which comprises a step of applying an effective amount of the invented compound to plants and soils (hereinafter referred to as “invented controlling method”).

The present invention further provides an amide compound represented by the following formula (V) or a salt thereof used for preparation of the invented compound (hereinafter referred to as “present amide compound”):

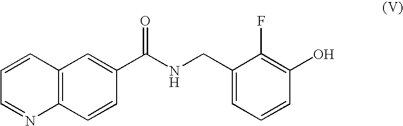

Furthermore, the present invention provides an amine compound represented by the following formula (VII) or a salt thereof (hereinafter referred to as “present amine compound”:

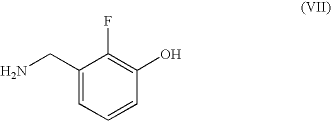

### ADVANTAGES OF THE INVENTION

The invented compound has excellent control effect for plant diseases, and hence is useful as an active ingredient of plant disease controlling agents.

### BEST MODE FOR CARRYING OUT THE INVENTION

A process for producing the invented compound will be explained.

The invented compound can be produced, for example, by the following (process 1)-(process 3).

### (Process 1)

The invented compound can be produced by causing compound (II) to react with compound (III) or a salt thereof (e.g., hydrochloride and hydrobromide) in the presence of a dehydration condensation agent:

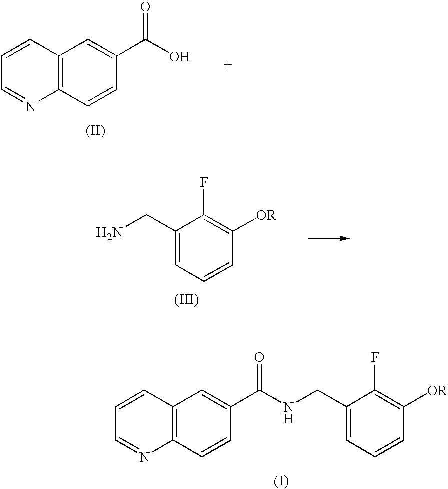

(in the formulas, R represents a C1-C5 straight-chain alkyl group).

The above reaction is carried out usually in the presence of a solvent.

As the solvent used for the reaction, mention may be made of, for example, ethers such as tetrahydrofuran (hereinafter sometimes referred to as “THF”), ethylene glycol dimethyl ether and tert-butylmethyl ether (hereinafter sometimes referred to as “MTBE”), aliphatic hydrocarbons such as hexane, heptane and octane, aromatic hydrocarbons such as toluene and xylene, halogenated hydrocarbons such as chlorobenzene, esters such as butyl acetate and ethyl acetate, nitriles such as acetonitrile, acid amides such as N,N-dimethylformamide (hereinafter sometimes referred to as “DMF”), sulfoxides such as dimethyl sulfoxide (hereinafter sometimes referred to as DMSO″), nitrogen-containing aromatic compounds such as pyridine, and mixtures thereof.

The dehydration condensation agents used for the reaction include, for example, carbodiimides such as 1-ethyl-3-(3-dimethylaminopropyl)carbodiimide hydrochloride (hereinafter referred to as “WSC”) and 1,3-dicyclohexylcarbodiimide, (benzotriazol-1-yloxy)tris(dimethylamino)phosphonium hexafluorophosphate (hereinafter sometimes referred to as “BOP reagent”.

For 1 mole of the compound (II), the compound (III) is used usually in a proportion of 0.5-3 moles and the dehydration condensation agent is used usually in a proportion of 1-5 moles.

The reaction temperature is usually from −20° C. to +140° C., and the reaction time is usually 1-24 hours.

After completion of the reaction, water is added, and when solid is precipitated, filtration is carried out, whereby the invented compound can be isolated, and when no solid is precipitated, the reaction mixture is subjected to post-treatments such as extraction with an organic solvent and drying and concentration of the organic layer, etc., whereby the invented compound can be isolated. The isolated invented compound can be further purified by chromatography, re-crystallization, or the like.

### (Process 2)

The invented compound can be produced by causing compound (IV) or a salt thereof (e.g., a hydrochloride) to react with compound (III) or a salt thereof (e.g., hydrochloride and hydrobromide) in the presence of a base:

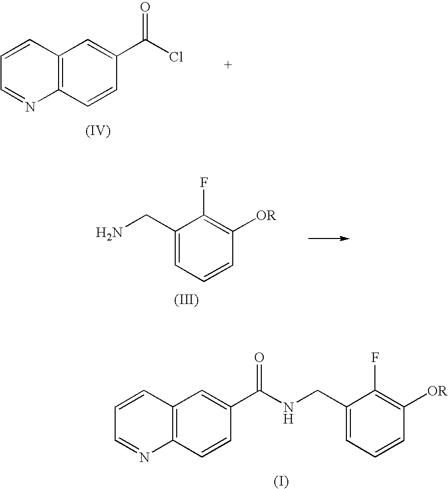

(in the formulas, R is as defined above).

The above reaction is carried out usually in the presence of a solvent.

As the solvent used for the reaction, mention may be made of, for example, ethers such as THF, ethylene glycol dimethyl ether and MTBE, aliphatic hydrocarbons such as hexane, heptane and octane, aromatic hydrocarbons such as toluene and xylene, halogenated hydrocarbons such as chlorobenzene, esters such as butyl acetate and ethyl acetate, nitriles such as acetonitrile, and mixtures thereof.

The bases used for the reaction include, for example, alkali metal carbonates such as sodium carbonate and potassium carbonate, tertiary amines such as triethylamine and diisopropylethylamine, and nitrogen-containing aromatic compounds such as pyridine and 4-dimethylaminopyridine.

For 1 mole of the compound (IV), the compound (III) is used usually in the proportion of 0.5-3 moles and the base is used usually in the proportion of 1-5 moles.

The reaction temperature is usually from −20° C. to +100° C., and the reaction time is usually 0.1-24 hours.

After completion of the reaction, water is added, and when solid is precipitated, filtration is carried out, whereby the invented compound can be isolated, and when no solid is precipitated, the reaction mixture is subjected to post-treatments such as extraction with an organic solvent and drying and concentration of the organic layer, etc., whereby the invented compound can be isolated. The isolated invented compound can be further purified by chromatography, re-crystallization, or the like.

### (Process 3)

The invented compound can be produced by causing the present amide compound (V) to react with the following compound (VI) in the presence of a base:

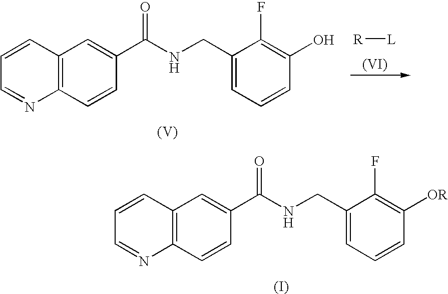

(in the formulas, R is as defined above, and L represents a chlorine atom, a bromine atom, an iodine atom, a methanesulfonyloxy group, a trifluoromethanesulfonyloxy group or a p-toluenesulfonyloxy group).

The above reaction is carried out usually in the presence of a solvent.

As the solvent used for the reaction, mention may be made of, for example, ethers such as THF, ethylene glycol dimethyl ether and MTBE, aromatic hydrocarbons such as toluene and xylene, halogenated hydrocarbons such as chlorobenzene, nitriles such as acetonitrile, acid amides such as DMF, sulfoxides such as dimethyl sulfoxide, ketones such as acetone, methyl ethyl ketone and methyl isobutyl ketone, water and mixtures thereof.

The bases used for the reaction include, for example, alkali metal carbonates such as sodium carbonate, potassium carbonate and cesium carbonate, alkali metal hydroxides such as sodium hydroxide, and alkali metal hydrides such as sodium hydride.

For 1 mole of the present amide compound (V), usually, the compound (VI) is used in the proportion of 1-10 moles and the base is used in the proportion of 1-5 moles.

The reaction temperature is usually from −20° C. to +100° C., and the reaction time is usually 0.1-24 hours.

After completion of the reaction, water is added, and when solid is precipitated, filtration is carried out, whereby the invented compound can be isolated, and when no solid is precipitated, the reaction mixture is subjected to post-treatments such as extraction with an organic solvent and drying and concentrating of the organic layer, whereby the invented compound can be isolated. The isolated invented compound can be further purified by chromatography, re-crystallization, or the like.

A process for producing the present amide compound (V) will be explained.

The present amide compound (V) can be produced, for example, by the following (synthesis process 1) or (synthesis process 2).

### (Synthesis Process 1)

The present amide compound (V) can be produced by causing compound (II) to react with compound (VII) or a salt thereof (e.g., hydrochloride and hydrobromide) in the presence of a dehydration condensation agent.

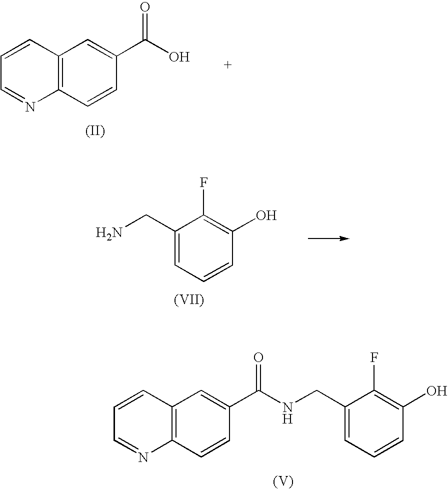

The above reaction is carried out usually in the presence of a solvent.

As the solvent used for the reaction, mention may be made of, for example, ethers such as THF, ethylene glycol dimethyl ether and MTBE, aliphatic hydrocarbons such as hexane, heptane and octane, aromatic hydrocarbons such as toluene and xylene, halogenated hydrocarbons such as chlorobenzene, esters such as butyl acetate and ethyl acetate, nitriles such as acetonitrile, acid amides such as DMF, sulfoxides such as DMSO, nitrogen-containing aromatic compounds such as pyridine, and mixtures thereof.

The dehydration condensation agents used for the reaction include, for example, carbodiimides such as WSC and 1,3-dicyclohexylcarbodiimide, and BOP reagent.

For 1 mole of the compound (II), usually the compound (VII) is used in a proportion of 0.5-3 moles and the dehydration condensation agent is used in a proportion of 1-5 moles.

The reaction temperature is usually from −20° C. to +140° C., and the reaction time is usually 1-24 hours.

After completion of the reaction, water is added, and when solid is precipitated, filtration is carried out, whereby the present amide compound can be isolated, and when no solid is precipitated, the reaction mixture is subjected to after-treatments such as extraction with an organic solvent, and drying and concentration of the organic layer, whereby the present amide compound can be isolated. The isolated present amide compound can be further purified by chromatography, re-crystallization, or the like.

### (Synthesis Process 2)

The present amide compound (V) can be produced by causing compound (IV) or a salt thereof (e.g., hydrochloride) to react with compound (VII) or a salt thereof (e.g., hydrochloride and hydrobromide) in the presence of a base.

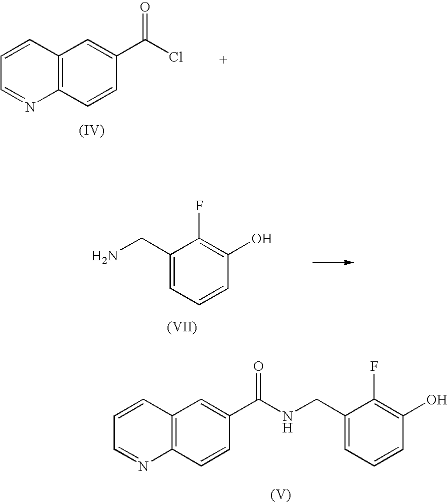

The above reaction is carried out usually in the presence of a solvent.

As the solvent used for the reaction, mention may be made of, for example, ethers such as THF, ethylene glycol dimethyl ether and MTBE, aliphatic hydrocarbons such as hexane, heptane and octane, aromatic hydrocarbons such as toluene and xylene, halogenated hydrocarbons such as chlorobenzene, esters such as butyl acetate and ethyl acetate, nitriles such as acetonitrile, and mixtures thereof.

The bases used for the reaction include, for example, alkali metal carbonates such as sodium carbonate and potassium carbonate, tertiary amines such as triethylamine and diisopropylethylamine, and nitrogen-containing aromatic compounds such as pyridine and 4-dimethylaminopyridine.

For 1 mole of the compound (IV), usually the compound (VII) is used in a proportion of 0.5-1 mole and the base is used in a proportion of 1-5 moles.

The reaction temperature is usually from −20° C. to +100° C., and the reaction time is usually 0.1-24 hours.

After completion of the reaction, water is added, and when solid is precipitated, filtration is carried out, whereby the present amide compound can be isolated, and when no solid is precipitated, the reaction mixture is subjected to after-treatments such as extraction with an organic solvent, and drying and concentration of the organic layer, whereby the present amide compound can be isolated. The isolated present amide compound can be further purified by chromatography, re-crystallization, or the like.

Next, a process for producing the present amine compound (VII) will be explained.

The present amine compound (VII) can be produced, for example, by the following (synthesis process).

### (Synthesis Process)

The present amine compound (VII) can be produced by demethylation of methoxy group of compound (III-1).

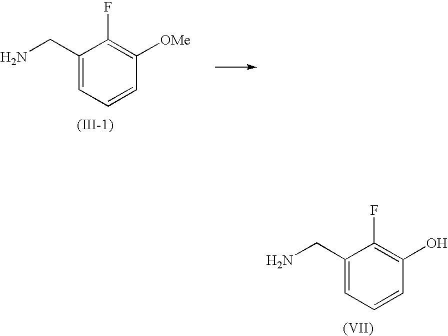

For example, when methoxy group is subjected to demethylation with an acid, this reaction can be carried out in the presence of a solvent, e.g., an alcohol solvent such as methanol, ethanol or isopropyl alcohol, an organic acid solvent such as acetic acid or trifluoroacetic acid, or water, and can also be carried out in the absence of solvent.

Examples of the acid used for the reaction are inorganic acids such as hydrochloric acid, hydrobromic acid and sulfuric acid.

The acid is usually used in a proportion of 2-20 moles for 1 mole of the compound (III-1).

The reaction temperature is usually 0-150° C., and the reaction time is usually 0.1-24 hours.

After completion of the reaction, the compound (VII) can be obtained as a salt by concentrating the reaction mixture.

The compound (II), some species of the compound (III) or salts thereof, and the compound (IV) or a salt thereof used for preparation of the invented compound are compounds which are commercially available or disclosed in literatures.

The compound (III) can be produced in accordance with, for example, the process disclosed in Journal of Organic Chemistry, Vol. 53, No. 5, pp. 1064-1071, 1988. U.S. Pat. No. 4,594,092 discloses that the compound (III-1) which is the compound (III) where R is a methyl group was suspected to be able to be used as a starting material, but makes no mention of specific production process and physical properties:

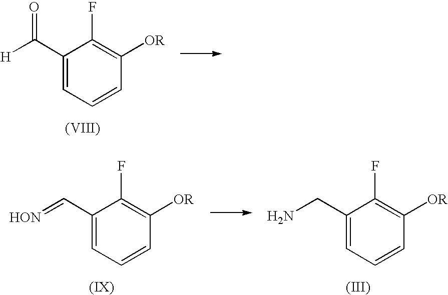

(in the formulas, R is as defined above).

Examples of the invented compounds are N-(2-fluoro-3-methoxyphenyl)methyl-quinoline-6-carboxamide, N-(3-ethoxy-2-fluorophenyl)methyl-quinoline-6-carboxamide, N-(2-fluoro-3-propoxyphenyl)methyl-quinoline-6-carboxamide, N-(3-butoxy-2-fluorophenyl)methyl-quinoline-6-carboxamide, and N-(2-fluoro-3-pentyloxyphenyl)methyl-quinoline-6-carboxamide.

Although the invented controlling agent may be composed of only the invented compound, the invented compound is used usually after having been formulated into any of formulations such as wettable powders, water dispersible granules, flowable concentrates, granules, dry flowable concentrates, emulsifiable concentrates, aqueous liquid formulations, oil formulations, smoking formulations, aerosols, microcapsules, etc. by mixing with a carrier (e.g. a solid, liquid or gaseous carrier), a surfactant and other auxiliaries for formulation, such as adhesive agent, dispersant, stabilizer, etc. These formulations contain the invented compound in a proportion of usually 0.1 to 99% by weight, preferably 0.2 to 90% by weight.

The solid carriers used for formulation include, for example, fine powders or particles of clays (e.g., kaolin, diatomaceous earth, synthetic hydrous silicon oxide, agalmatolite clay, bentonite, acid clay, and talc), and other inorganic minerals (e.g., sericite, quartz powder, sulfur powder, activated carbon, calcium carbonate, and hydrated silica), liquid carriers include, for example, water, alcohols (e.g., methanol, and ethanol), ketones (e.g., acetone, and methyl ethyl ketone), aromatic hydrocarbons (e.g., benzene, toluene, xylene, ethylbenzene, and methylnaphthalene), aliphatic or alicyclic hydrocarbons (e.g., n-hexane, cyclohexanone, and kerosene), esters (e.g., ethyl acetate, and butyl acetate), nitriles (e.g., acetonitrile, and isobutyronitrile), ethers (e.g., dioxane, and diisopropyl ether), acid amides (e.g., dimethylformamide, and dimethylacetamide), and halogenated hydrocarbons (e.g., dichloroethane, trichloroethylene, and carbon tetrachloride).

The surfactant includes, for example, alkyl sulfates, alkylsulfonates, alkylarylsulfonates, alkyl aryl ethers and their polyoxyethylenated products, polyoxyethylene glycol ethers, polyhydric alcohol esters and sugar alcohol derivatives.

The other auxiliaries for formulation include, for example, adhesive agents, dispersants, thickening agents, wetting agents, extending agents and antioxidants. Specific examples thereof are casein, gelatin, polysaccharides (e.g. starch, gum arabic, cellulose derivative and alginic acid), lignin derivatives, bentonite, saccharides, synthetic water-soluble polymers [e.g. poly(vinyl alcohols, poly(vinylpyrrolidone)s and poly(acrylic acid)s], PAP (acidic isopropyl phosphate), BHT (2,6-di-tert-butyl-4-methylphenol), BHA (a mixture of 2-tert-butyl-4-methoxyphenol and 3-tert-butyl-4-methoxyphenol), vegetable oils, mineral oils, fatty acids, their esters, etc.

A method for applying the invented controlling agent in order to control plant diseases is not particularly limited. The method includes, for example, treatment of plants, such as foliage application; treatment of a plant cultivation area, such as soil treatment; and treatment of seeds, such as seed disinfection.

The invented controlling agent can be used in admixture with other fungicides, insecticides, acaricides, nematicides, herbicides, plant growth regulators, fertilizers or soil conditioners. It is also possible to use the invented controlling agent in combination with such other chemicals without mixing with them.

Such other fungicides include, for example, azole fungicidal compounds such as propiconazole, prothioconazole, triadimenol, prochloraz, penconazole, tebuconazole, flusilazole, diniconazole, bromuconazole, epoxiconazole, difenconazole, cyproconazole, metconazole, triflumizole, tetraconazole, myclobutanil, fenbuconazole, hexaconazole, fluquinconazole, triticonazole, bitertanol, imazalil, flutriafol, etc.; cyclic amine fungicidal compounds such as fenpropimorph, tridemorph, fenpropidin, etc.; benzimidazole fungicidal compounds such as carbendazim, benomyl, thiabendazole, thiophanate-methyl, etc.; procymidone, cyprodinil, pyrimethanil, diethofencarb, thiuram, fluazinam, mancozeb, iprodione, vinclozolin, chlorothalonil, captan, mepanipyrim, fenpiclonil, fludioxonil, dichlofluanid, folpet, kresoxim-methyl, azoxystrobin, trifloxystrobin, fluoxastrobin, picoxystrobin, pyraclostrobin, dimoxystrobin, pyribencarb, spiroxamine, quinoxyfen, fenhexamid, famoxadone, fenamidone, zoxamide, ethaboxam, amisulbrom, iprovalicarb, benthiavalicarb, cyazofamid, mandipropamid, boscalid, penthiopyrad, metrafenone, fluopyram, bixafen, cyflufenamid and proquinazid.

Although the applying dosage of the invented controlling agent is varied depending on weather conditions, formulation, when, where and how the invented controlling agent is applied, diseases to be controlled, crop plants to be protected, etc., it is usually 1 to 500 g, preferably 2 to 200 g, (in terms of the invented compound in the invented controlling agent), per 10 ares. When the invented controlling agent is an emulsifiable concentrate, wettable powder, suspension concentrate or the like, it is usually applied after having been diluted with water. In this case, the concentration of the invented compound after the dilution is usually 0.0005 to 2% by weight, preferably 0.005 to 1% by weight. When the invented controlling agent is a powder, granules or the like, it is applied as it is without dilution. When the invented controlling agent is applied to seeds, its applying dosage is usually 0.001 to 100 g, preferably 0.01 to 50 g, (in terms of the invented compound in the invented controlling agent), per Kg of the seeds.

The invented controlling agent can be used as a composition for controlling plant diseases in crop lands such as upland field, paddy field, lawn and turf, orchard, etc. The present controller can control plant diseases in crop lands where the following “crops” or the like are cultivated.

Field crops: corn, rice, wheat, barley, rye, oat, sorghum, cotton, soybean, peanut, buckwheat, beet, rape, sunflower, sugar cane, tobacco, etc.

Vegetables: Solanaceae (e.g. eggplant, tomato, green pepper, pepper and potato), Cucurbotaceae (e.g. cucumber, pumpkin, zucchini, watermelon and melon), Cruciferae (e.g. Japanese radish, turnip, horseradish, kohlrabi, Chinese cabbage, cabbage, leaf mustard, broccoli and cauliflower), Compositae (e.g. edible burdock, garland chrysanthemum, globe artichoke and lettuce), Liliacede (e.g. Welsh onion, onion, garlic and asparagus), Umbelliferae (e.g. carrot, parsley, celery and Pstinaca), Chenopodiales (e.g. spinach and chard), Lamiaceae (e.g. perilla, mint and basil), strawberry, sweet potato, Chinese yam, taro, etc.

Flowers and ornament plants.

Ornamental foliage plants.

Fruit trees: pomaceous fruits (e.g. apple, pear, Japanese pear, Chinese quince and quince), stone fruits (e.g. peach, plum, nectarine, Japanese apricot, cherry, apricot and prune), citrus fruits (e.g. Satsuma mandarin, orange, lemon, lime and grapefruit), nut trees (e.g. chestnut, walnut, hazel, almond, pistachio, cashew nut and macadamia nut), berries (e.g. blueberry, cranberry, blackberry and raspberry), grape, Japanese persimmon, olive, loquat, banana, coffee, date palm, coconut palm, etc.

Trees other than fruit trees: tea, mulberry, flowering trees and shrubs, street trees (Japanese ash, birch, flowering dogwood, blue gum, ginkgo, lilac, maple, oak, poplar, Chinese redbud, Formosa sweet gum, plane trees, zelkova, Japanese arborvitae, fir, Japanese hemlock, needle juniper, pine, Japanese spruce and Japanese yew).

The above-mentioned “crops” also include crops having resistance to herbicides such as HPPD inhibitors (e.g. isoxaflutole), ALS inhibitor (e.g. imazethapyr and thifensulfuron-methyl), EPSP synthetase inhibitors, glutamine synthetase inhibitors, bromoxynil, dicamba, etc. which has been imparted by a classic breeding method or a genetic recombination technology.

Examples of the “crops” having the resistance imparted by the classic breeding method include Clearfield Canola® resistant to imidazolinone herbicides (e.g. imazethapyr) and STS soybean resistant to sulfonylurea ALS inhibition type herbicides (e.g. thifensulfuron-methyl). As crops having the resistance imparted by the genetic recombination technology, corn cultivars resistant to glyphosate and glufosinato are exemplified and are already on the market under the trade names of RoundupReady®, RoundupReady 2® and LibertyLink®.

The above-mentioned “crops” also include crops which a genetic recombination technology has enabled to synthesize a selective toxin known in the case of, for example, Bacillus.

Examples of toxins produced in such genetically modified plants include insecticidal proteins derived from Bacillus cereus and Bacillus popilliae; insecticidal proteins such as δ-endotoxins (e.g. Cry1Ab, Cry1Ac, Cry1F, Cry1Fa2, Cry2Ab, Cry3A, Cry3Bb1 and Cry9C), VIP1, VIP2, VIP3, VIP3A, etc., which are derived from Bacillus thuringiensis; toxins derived from nematodes; toxins produced by animals, such as scorpion toxin, spider toxin, bee toxin, insect-specific neurotoxins, etc.; filamentous fungi toxins; plant lectins; agglutinin; protease inhibitors such as trypsin inhibitors, serine protease inhibitors, patatin, cystatin, papain inhibitors, etc.; ribosome-inactivating proteins (RIP) such as ricin, corn-RIP, abrin, rufin, sapolin, priodin, etc.; steroid metabolic enzymes such as 3-hydroxysteroid oxidase, ecdysteroid-UDP-glucosyltransferase, cholesterol oxidase, etc.; ecdysone inhibitors; HMG-COA reductase; ion channel inhibitors such as sodium channel inhibitors, calcium channel inhibitors, etc.; juvenile hormone esterase; diuretic hormone receptors; stilbene synthetase; bibenzyl synthetase; chitinase; and glucanase.

The toxins produced in such genetically modified crops also include hybrid toxins, partly deficient toxins and modified toxins of insecticidal proteins such as δ-endotoxin proteins (e.g. Cry1Ab, Cry1Ac, Cry1F, Cry1Fa2, Cry2Ab, Cry3A, Cry3Bb1 and Cry9C), VIP1, VIP2, VIP3, VIP3A, etc. The hybrid toxins are produced by a novel combination of the different domains of such a protein by adopting a recombination technology. As the partly deficient toxin, Cry1Ab deficient in a part of the amino acid sequence is known. In the modified toxins, one or more amino acids of a natural toxin have been replaced.

Examples of such toxins and genetically modified plants capable of synthesizing such toxins are described in EP-A-0 374 753, WO 93/07278, WO95/34656, EP-A-0 427 529, EP-A-451 878, WO 03/052073, etc.

The toxins contained in such genetically modified plants impart resistance to insect pests of Coleoptera, insect pests of Diptera and insect pests of Lepidoptera to the plants.

Genetically modified plants containing one or more insecticidal insect-resistant genes and capable of producing one or more toxins have already been known, and some of them are on the market. Examples of such genetically modified plants include YieldGard® (a corn cultivar capable of producing Cry1Ab toxin), YieldGard Rootworm® (a corn cultivar capable of producing Cry3Bb1 toxin), YieldGard Plus® (a corn cultivar capable of producing Cry1Ab and Cry3Bb1 toxins), Herculex I® (a corn cultivar capable of producing phosphinotrysin N-acetyltransferase (PAT) for imparting resistance to Cry1Fa2 toxin and Glyfosinate), NuCOTN33B (a cotton cultivar capable of producing Cry1Ac toxin), Bollgard I® (a cotton cultivar capable of producing Cry1Ac toxin), Bollgard II® (a cotton cultivar capable of producing Cry1Ac and Cry2Ab toxins), VIPCOT® (a cotton cultivar capable of producing VIP toxin), NewLeaf® (a potato cultivar capable of producing Cry3A toxin), NatureGard®, Agrisure®, CB Advantage (Bt11 corn borer (CB) properties), and Protecta®.

The above-mentioned “crops” also include crops having an ability to produce an anti-pathogenic substance having selective action which has been imparted by a gene recombination technology.

As examples of the anti-pathogenic substance, PR proteins and the like are known (PRPs, EP-A-0 392 225). Such anti-pathogenic substances and genetically modified plants capable of producing them are described in EP-A-0 392 225, WO 95/33818, EP-A-0 353 191, etc.

Examples of such anti-pathogenic substances produced by the genetically modified plants include ion channel inhibitors such as sodium channel inhibitors, calcium channel inhibitors (for example, KP1, KP4 and KP6 toxins produced by viruses are known), etc.; stilbene synthases; bibenzyl synthases; chitinase; glucanase; PR proteins; and anti-pathogenic substances produced by microorganisms, such as peptide antibiotics, antibiotics having a heterocyclic ring, protein factors concerned in resistance to plant diseases (which are called plant-disease-resistant genes and are described in WO 03/000906), etc.

The above-mentioned “crops” also include crops having two or more properties relating to the above-mentioned herbicide resistance, insect pest resistance, disease resistance and the like, which have been imparted by a classic breeding technique or a genetic recombination technology; and crops having two or more properties derived from parents which have been imparted by mating between genetically modified plants having the same or different properties.

Plant diseases controllable by the present invention include, for example, fungal diseases. More particularly, the diseases described below can be exemplified as the plant diseases. The plant diseases are not limited to them.

The invented controlling method is usually practiced by applying the invented controlling agent by the above-mentioned method for applying the invented controlling agent.

Blast (Magnaporthe grisea), Helminthosporium leaf spot (Cochliobolus miyabeanus), sheath blight (Rhizoctonia solani) and “Bakanae” disease (Gibberella fujikuroi) of rice; powdery mildew (Erisiphe graminis), scab (Fusarium graminearum, F. avenacerum, F. culmorum, Microdochium nivale), rust (Puccinia striiformis, P. graminis, P. recondite, P. hordei), snow blight (Typhula sp., Micronectriella nivalis), loose smut (Ustilago tritici, U. nude), bunt (Tilletia caries), eyespot (Pseudocercosporella herpotrichoides), scald (Rhynchosporium secalis), leaf blight (Septoria tritici), glume blotch (Leptosphaeria nodorum), net blotch (Pyrenophora teres Drechaler) and take-all (Gaeumannomyces graminis) of barley, wheat, oats and rye; melanose (Diaporthe citri), scab (Elsinoe fawcetti) and penicillium rot (Penicillium digitatum, P. italicum) of citrus; blossom blight (Monilinia mali), canker (Valsa ceratosperma), powdery mildew (Podosphaera leucotricha), Alternaria leaf spot (Alternaria altenate apple pathotype), scab (Venturia inaqualis) and anthracnose (Glomerella cingulata) of apple; scab (Venturia nashicola, V. pirina), black spot (Alternaria alternate Japanese pear pathotype) and rust (Gymnosporangium haraeanum) of pear; brown rot (Monilinia fructicola), scab (Cladosporium carpophilum) and Phomopsis rot (Phomopsis sp of peach; anthracnose (Elsinoe ampelina), ripe rot (Glomerella cingulata), powdery mildew (Uncinula necator), rust (Phakopsora ampelopsidis), black rot (Guignardia bidwellii) and downy mildew (Plasmopara viticola) of grape; anthracnose (Gloeosporium kaki) and leaf spot (Cercospora Mycosphaerella nawae) of Japanese persimmon; anthracnose (Colletotrichum lagenarium), powdery mildew (Sphaerotheca fuliginea), gummy stem blight (Mycosphaerella melonis), stem rot (Fusarium oxyspoxum), downy mildew (Pseudoperonospora cubensis), Phytophthora rot (Phytophthora sp.) and seedling blight (Phthium sp.) of melons and cucumber; early blight (Alternaria solani), leaf mold (Cladosporium fulvum) and late blight (Phytophthora infestans) of tomato; brown spot (Phomopsis vexans) and powdery mildew (Erisiphe cichoracearum) of eggplant; alternaria leaf spot (Alternaria japonica) and white spot (Cercosporella brassicae) of vegetables of Crusiferae; Welsh onion rust (Puccinia allii); purple stain (Cercospora kikuchii), Sphaceloma scab (Elsinoe glycines), pod and stem blight (Diaporthe phaseolorum var. sojae) and rust (Phakopsora pachyrhizi) of soybean; kidney bean anthracnose (Colletotrichum lindemthianum); leaf spot (Cercospora personata), leaf spot (Cercospora arachidicola) and southern blight (Sclerotium rolfsii) of peanut; pea powdery mildew (Erisiphe pisi); early blight (Alternaria solani), late blight (Phytophthora infestans) and Verticillium wilt (Verticillium albo-atrum, V. dahliae, V. nigrescens) of potato; strawberry powdery mildew (Sphaerotheca humuli); net blister blight (Exobasidium reticulatum), white scab (Elsinoe leucospila), zonate leaf spot (Pestalotiopsis sp.) and anthracnose (Colletotrichum theae-sinensis) of tea plant; brown spot (Alternaria longipes), powdery mildew (Erysiphe cichoracearum), anthracnose (Colletotrichum tabacum), downy mildew (Peronospora tabacina), Phytophthora rot (Phytophthora nicotianae) of tobacco;

leaf spot (Cercospora beticola), foliage blight (Thanatephorus cucumeris) and root rot (Thanatephorus cucumeris) of beet; black spot (Diplocarpon rosae) and powdery mildew (Sphaerotheca pannosa) of rose; leaf blight (Septoria chrysanthemi-indici) and white rust (Puccinia horiana) of chrysanthemum; Botrytis diseases (Botrytis cinerea, B. byssoidea, B. squamosa), gray mold neck rot (Botrytis alli) and Small sclerotial neck rot (Botrytis squamosa) of onion; gray mold (Botrytis cinerea) and stem rot (Sclerotinia sclerotiorum) of various crops; Alternaria leaf spot (Alternaria brassicicola) of Japanese radish; dollar spot (Sclerotinia homeocarpa), brown patch and large patch (Rhizoctonia solani) of turf grass; and Sigatoka diseases (Mycosphaerella fijiensis, Mycosphaerella musicola, Pseudocercospora musae) of banana.

### EXAMPLES

The present invention will be explained in more detail by preparation examples, formulation examples and test examples, which should not be construed as limiting the invention. The “part” is by weight.

### Preparation Example 1 of the Invented Compound

0.21 g of WSC was added to a mixture of 0.17 g of 6-quinolinecarboxylic acid, 0.16 g of 2-fluoro-3-methoxybenzylamine hydrochloride and 3 ml of pyridine, followed by stirring at room temperature for 12 hours. Water was added to the reaction mixture, and precipitated solid was collected by filtration. The collected solid was washed with saturated aqueous sodium hydrogencarbonate solution, water and hexane in succession and dried to obtain 0.24 g of N-(2-fluoro-3-methoxyphenyl)methyl-quinoline-6-carboxamide (hereinafter referred to as “invented compound (1)”).

The invented compound (1):

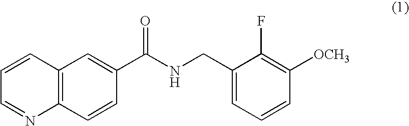

¹H-NMR (CDCl₃) δ: 3.90 (3H, s), 4.77 (2H, d, J=5.6 Hz), 6.70 (1H, br s), 6.92-6.96 (1H, m), 7.02-7.10 (2H, m), 7.47 (1H, dd, J=8.1, 4.0 Hz), 8.06 (1H, d, J=8.7 Hz), 8.15 (1H, d, J=8.7 Hz), 8.23 (1H, d, J=8.1 Hz), 8.31 (1H, s), 8.98 (1H, d, J=4.0 Hz).

### Preparation Example 2 of the Invented Compound

0.25 g of cesium carbonate was added to a mixed solution of 0.17 g of N-(2-fluoro-3-hydroxyphenyl)methyl-quinoline-6-carboxamide, 0.12 g of iodoethane and 3 ml of DMF, followed by stirring at room temperature for 12 hours. Water was added to the reaction mixture, and precipitated solid was collected by filtration. The collected solid was washed with 15% aqueous sodium hydroxide solution, water and hexane in succession and dried to obtain 0.10 g of N-(3-ethoxy-2-fluorophenyl)methyl-quinoline-6-carboxamide (hereinafter referred to as “invented compound (2)”).

The invented compound (2):

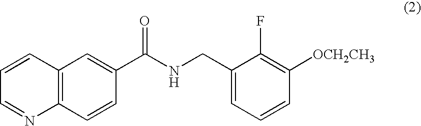

¹H-NMR (CDCl₃) δ: 1.47 (3H, t, J=7.0 Hz), 4.12 (2H, q, J=7.0 Hz), 4.76 (2H, d, J=5.8 Hz), 6.70 (1H, br s), 6.91-6.95 (1H, m), 7.00-7.08 (2H, m), 7.48 (1H, dd, J=8.3, 4.1 Hz), 8.06 (1H, dd, J=8.8, 1.7 Hz), 8.15 (1H, d, J=8.8 Hz), 8.24 (1H, d, J=8.3 Hz), 8.31 (1H, d, J=1.7 Hz), 8.99 (1H, dd, J=4.1, 1.7 Hz).

### Preparation Example 3 of the Invented Compound

0.25 g of cesium carbonate was added to a mixed solution of 0.17 g of N-(2-fluoro-3-hydroxyphenyl)methyl-quinoline-6-carboxamide, 0.16 g of 1-iodopropnane and 3 ml of DMF, followed by stirring at room temperature for 12 hours. Water was added to the reaction mixture, and precipitated solid was collected by filtration. The collected solid was washed with 15% aqueous sodium hydroxide solution, water and hexane in succession and dried to obtain 0.11 g of N-(2-fluoro-3-propoxyphenyl)methyl-quinoline-6-carboxamide (hereinafter referred to as “invented compound (3)”).

The invented compound (3):

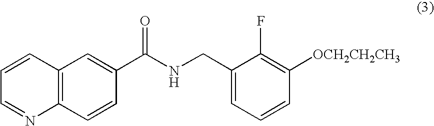

¹H-NMR (CDCl₃) δ: 1.06 (3H, t, J=7.4 Hz), 1.81-1.90 (2H, m), 4.00 (2H, t, J=6.6 Hz), 4.76-4.77 (2H, m), 6.67 (1H, br s), 6.91-6.95 (1H, m), 7.00-7.07 (2H, m), 7.46-7.49 (1H, m), 8.06 (1H, d, J=8.8 Hz), 8.15 (1H, d, J=8.5 Hz), 8.24 (1H, d, J=7.6 Hz), 8.31 (1H, s), 8.98-8.99 (1H, m).

### Preparation Example 4 of the Invented Compound

0.25 g of cesium carbonate was added to a mixed solution of 0.17 g of N-(2-fluoro-3-hydroxyphenyl)methyl-quinoline-6-carboxamide, 0.14 g of 1-iodobutane and 3 ml of DMF, followed by stirring at room temperature for 12 hours. Water was added to the reaction mixture, and the precipitated solid was collected by filtration. The collected solid was washed with 15% aqueous sodium hydroxide solution, water and hexane in succession and dried to obtain 0.15 g of N-(3-butoxy-2-fluorophenyl)methyl-quinoline-6-carboxamide (hereinafter referred to as “invented compound (4)”).

The invented compound (4):

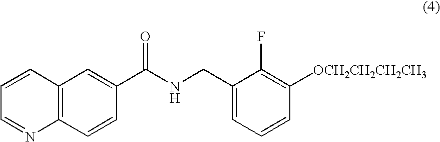

¹H-NMR (CDCl₃) δ: 0.99 (3H, t, J=7.4 Hz), 1.49-1.57 (2H, m), 1.78-1.85 (2H, m), 4.04 (2H, t, J=6.6 Hz), 4.76 (2H, d, J=5.6 Hz), 6.64 (1H, br s), 6.91-7.07 (3H, m), 7.47 (1H, dd, J=8.3, 4.1 Hz), 8.06 (1H, dd, J=8.8, 2.0 Hz), 8.15 (1H, d, J=8.8 Hz), 8.23-8.26 (1H, m), 8.31 (1H, d, J=1.7 Hz), 8.99 (1H, dd, J=4.1, 1.6 Hz).

### Preparation Example 5 of the Invented Compound

0.25 g of cesium carbonate was added to a mixed solution of 0.15 g of N-(2-fluoro-3-hydroxyphenyl)methyl-quinoline-6-carboxamide, 0.13 g of 1-iodopentane and 3 ml of DMF, followed by stirring at room temperature for 12 hours. Water was added to the reaction mixture, and the precipitated solid was collected by filtration. The collected solid was washed with 15% aqueous sodium hydroxide solution, water and hexane in succession and dried to obtain 0.13 g of N-(2-fluoro-3-pentyloxyphenyl)methyl-quinoline-6-carboxamide (hereinafter referred to as “invented compound (5)”).

The invented compound (5):

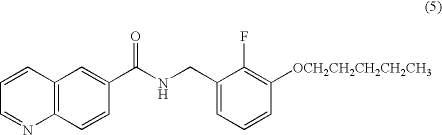

¹H-NMR (CDCl₃) δ: 0.93 (3H, t, J=6.5 Hz), 1.26-1.46 (4H, m), 1.76-1.84 (2H, m), 4.02 (2H, t, J=6.5 Hz), 4.75 (2H, d, J=5.1 Hz), 6.78 (1H, br s), 6.90-7.06 (3H, m), 7.45-7.48 (1H, m), 8.05 (1H, d, J=8.0 Hz), 8.14 (1H, d, J=8.3 Hz), 8.22 (1H, d, J=7.6 Hz), 8.30-8.31 (1H, m), 8.96-8.98 (1H, m).

### Synthesis Example of the Present Amide Compound

3.33 g of WSC was added to a mixture of 2.59 g of 6-quinoline carboxylic acid, 3.33 g of 2-fluoro-3-hydroxybenzylamine hydrobromide and 20 ml of pyridine, followed by stirring at room temperature for 12 hours. Water was added to the reaction mixture, and the precipitated solid was collected by filtration. The collected solid was washed with saturated aqueous sodium hydrogencarbonate solution, water and hexane in succession and dried to obtain 2.6 g of N-(2-fluoro-3-hydroxyphenyl)methyl-quinoline-6-carboxamide.

N-(2-fluoro-3-hydroxyphenyl)methyl-quinoline-6-carboxamide:

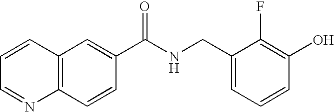

¹H-NMR (DMSO-d₆) δ: 1.70 (1H, s), 4.55-4.57 (2H, m), 6.75-6.93 (3H, m), 7.60-7.64 (1H, m), 8.10 (1H, d, J=8.0 Hz), 8.22 (1H, d, J=8.0 Hz), 8.48 (1H, d, J=7.3 Hz), 8.57 (1H, s), 8.99 (1H, s), 9.25 (1H, s).

### Synthesis Example of the Present Amine Compound

15 ml of 48% hydrobromic acid was added to 4 g of 2-fluoro-3-methoxybenzylamine hydrochloride, followed by refluxing with heating for 5 hours. The reaction mixture left to stand for cooling to about room temperature was concentrated under reduced pressure to obtain 4.0 g of 2-fluoro-3-hydroxybenzylamine hydrobromide.

2-fluoro-3-hydroxybenzylamine hydrobromide:

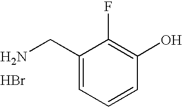

¹H-NMR (DMSO-d₆) δ: 4.04 (2H, q, J=5.5 Hz), 6.90-6.94 (1H, m), 6.97-7.06 (2H, m), 8.23 (3H, br s), 10.05 (1H, br s).

### Preparation Example 1 of Intermediate

4.5 g of 2-fluoro-3-methoxybenzyl alcohol, 2.9 ml of methansulfonyl chloride and 50 ml of THF were mixed and stirred at 0° C. To the mixture was dropped 6.0 ml of triethylamine, followed by stirring at 0° C. for 30 minutes and then at room temperature for 2 hours. Thereafter, ethyl acetate was added to the reaction mixture left to stand for cooling to about room temperature, and then the mixture was filtered by passing through Celite®. Water was added to the filtrate, followed by extracting with ethyl acetate. The organic layer was washed successively with water and saturated aqueous sodium chloride solution and dried over magnesium sulfate, and then concentrated under reduced pressure to obtain 6.9 g of (2-fluoro-3-methoxyphenyl)methyl-methanesulfonate. (2-fluoro-3-methoxyphenyl)methyl-methanesulfonate

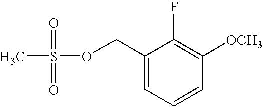

¹H-NMR (CDCl₃) δ: 3.00 (3H, s), 3.91 (3H, s), 5.30 (2H, s), 6.96-7.14 (3H, m).

### Preparation Example 2 of Intermediate

6.7 g of (2-fluoro-3-methoxyphenyl)methyl-methanesulfonate, 5.3 g of potassium phthalimide and 60 ml of DMF were mixed and stirred at 70° C. for 4 hours. Then, water was added to the reaction mixture, followed by extracting with ethyl acetate. The organic layer was washed successively with water, diluted hydrochloric acid and saturated aqueous sodium chloride solution and dried over magnesium sulfate, and then concentrated under reduced pressure. The resulting residue was washed with hexane to obtain 5.8 g of N-(2-fluoro-3-methoxyphenyl)methylphthalimide.

N-(2-fluoro-3-methoxyphenyl)methylphthalimide

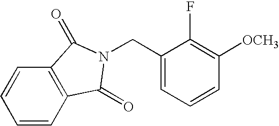

¹H-NMR (CDCl₃) δ: 3.87 (3H, s), 4.94 (2H, s), 6.86-6.90 (2H, m), 6.98-7.02 (1H, m), 7.73 (2H, dd, J=5.4, 3.0 Hz), 7.86 (2H, dd, J=5.4, 3.0 Hz).

### Preparation Example 3 of Intermediate

1.63 g of hydrazine monohydrate was dropped to a mixture of 7.7 g of N-(2-fluoro-3-methoxyphenyl)methylphthalimide and 30 ml of ethanol, followed by refluxing with heating for 4 hours. Then, the reaction mixture was cooled to room temperature and water was added thereto, and then the mixture was concentrated under reduced pressure. Diluted hydrochloric acid was added to the residue, followed by filtration. To the resulting filtrate was added ethyl acetate, and 15% aqueous sodium hydroxide solution was added thereto until the aqueous layer of the mixed solution became basic, followed by separation.

Concentrated hydrochloric acid was added to the resulting organic layer, and then the organic layer was concentrated under reduced pressure to obtain 4.5 g of 2-fluoro-3-methoxybenzylamine hydrochloride.

2-fluoro-3-methoxybenzylamine hydrochloride

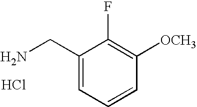

¹H-NMR (DMSO-d₆) δ: 3.85 (3H, s), 4.03 (2H, q, J=5.3 Hz), 7.13-7.23 (3H, m), 8.60 (3H, br s).

### Preparation Example 4 of Intermediate

24 ml of pyridine was dropped to a mixed solution of 15.4 g of 2-fluoro-3-methoxybenzaldehyde, 30 ml of THF and 3 ml of water, and thereto was added 13.8 g of hydroxylamine hydrochloride under ice cooling. The mixture was stirred at room temperature for 30 minutes, and concentrated under reduced pressure until the whole volume reduced to about half. Then, water was added to the resulting residue, followed by extracting with ethyl acetate. The organic layer was washed successively with diluted hydrochloric acid and saturated aqueous sodium chloride solution, and dried over magnesium sulfate, and then concentrated under reduced pressure to obtain 16 g of 2-fluoro-3-methoxybenzaldehyde oxime.

2-fluoro-3-methoxybenzaldehyde oxime

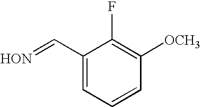

¹H-NMR (CDCl₃) δ: 3.90 (3H, s), 6.96-7.00 (1H, m), 7.03-7.12 (1H, m), 7.30-7.33 (1H, m), 7.64-7.78 (1H, m), 8.39 (1H, s).

### Preparation Example 5 of Intermediate

12.8 g of 2-fluoro-3-methoxybenzaldehyde oxime was added to a mixed solution of 2.4 g of 10% palladium carbon, 8.3 ml of 10N hydrochloric acid and 200 ml of ethanol, followed by stirring under normal pressure in hydrogen atmosphere. After absorption of hydrogen gas stopped, the reaction mixture was filtered through Celite®. The filtrate was concentrated under reduced pressure to obtain 8.2 g of 2-fluoro-3-methoxybenzylamine hydrochloride.

### Preparation Example 6 of Intermediate

A mixed solution of 10 g of 2-fluoro-3-methoxybenzaldehyde and 80 ml of ethanol was added to a mixed solution of 1.8 g of 10% palladium carbon, 10 ml of water, 6.5 ml of 10N hydrochloric acid and 50 ml of ethanol, and then 5.4 g of hydroxylamine hydrochloride was added thereto. The mixture was stirred at room temperature for 2 hours, followed by stirring under normal pressure in hydrogen atmosphere for 3 hours. The reaction mixture was filtered through Celite®, and the filtrate was concentrated under reduced pressure. To the residue was added 100 ml of water, followed by extracting with chloroform. 15% aqueous sodium hydroxide solution was added to the resulting aqueous layer to make it basic, followed by extracting with chloroform. The resulting organic layer was dried with potassium carbonate and concentrated under reduced pressure to obtain 8.0 g of 2-fluoro-3-methoxybenzylamine.

2-fluoro-3-methoxybenzylamine

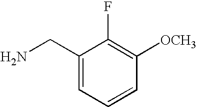

¹H-NMR (CDCl₃) δ: 1.55 (2H, br s), 3.89 (3H, s), 3.90 (2H, br s), 6.86-6.91 (2H, m), 7.02-7.06 (1H, m).

### Formulation Example 1

50 parts of one of the invented compounds (1)-(5), 3 parts of calcium ligninsulfonate, 2 parts of magnesium laurylsulfate, and 45 parts of synthetic hydrous silicon oxide are well ground and mixed to obtain a wettable powder.

### Formulation Example 2

20 parts of one of the invented compounds (1)-(5) and 1.5 part of sorbitan trioleate are mixed with 28.5 parts of an aqueous solution containing 2 parts of polyvinyl alcohol, and the mixture is pulverized by wet pulverizing method. Then, thereto is added 40 parts of an aqueous solution containing 0.05 part of xanthan gum and 0.1 part of aluminum magnesium silicate, and further added 10 parts of propylene glycol, followed by stirring and mixing to obtain a flowable formulation.

### Formulation Example 3

2 parts of one of the invented compounds (1)-(5), 88 parts of kaolin clay and 10 parts of talc are well ground and mixed to obtain a dust formulation.

### Formulation Example 4

5 parts of one of the invented compounds (1)-(5), 14 parts of polyoxyethylenestyrylphenyl ether, 6 parts of calcium dodecylbenzenesulfonate, and 75 parts of xylene are well mixed to obtain an emulsifiable concentrate.

### Formulation Example 5

2 parts of one of the invented compounds (1)-(5), 1 part of synthetic hydrous silicon oxide, 2 parts of calcium ligninsulfonate, 30 parts of bentonite and 65 parts of kaolin clay are well ground and mixed, then water is added thereto, followed by well kneading and granulation drying to obtain granule formulation.

### Formulation Example 6

10 parts of one of the invented compounds (1)-(5), 35 parts of white carbon containing 50 parts of polyoxyethylene alkyl ether sulfate ammonium salt and 55 parts of water are mixed, and the mixture is pulverized by wet pulverizing method to obtain a flowable formulation.

Next, usefulness of the invented compounds for controlling plant diseases is shown by test examples.

The area of lesions on the test plants at testing was visually observed, and the control effect was evaluated by comparing the area of lesions on test plants treated with the invented compound with the area of lesions on untreated plants.

As control test, N-(3-(3-methylbutoxy)phenyl)methyl-quinoline-6-carboxamide (represented by the following formula (A) and hereinafter referred to as “comparative compound (A)”) which is disclosed in Example E-29 of WO 2005/033079 was further used for test.

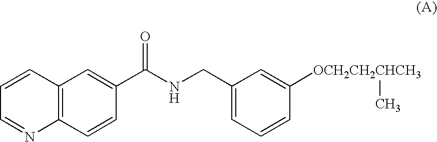

### Test Example 1

Test for showing preventive effect on Botrytis disease of cucumber (Botrytis cinerea):

Sand soil was packed in a plastic pot, and seeds of cucumber (variety: Sagami Hanjiro) were sowed therein and grown for 12 days in a greenhouse. Each of the invented compounds (1)-(5) and the comparative compound (A) was formulated into flowable formulation in accordance with Formulation Example 6, and it was diluted with water to a given concentration (200 ppm) and was sprayed onto the foliage of the cucumber so that a sufficient amount of the compound would be applied to the surface of leaves of the cucumber. After spraying, the plant was air-dried and PDA medium containing spores of Botrytis cinerea was placed on the surface of leaves of the cucumber. After the inoculation, the plant was left at 12° C. for 5 days under high humidity, and then the area of lesions was examined. As a result, the lesion area on the plant treated with the invented compounds (1)-(5) was less than 10% of the lesion area on the untreated plant. The lesion area on the plant treated with the comparative compound (A) was 98% of the untreated plant.

### Test Example 2

Test for showing curative effect on Botrytis disease of cucumber (Botrytis cinerea):

Sand soil was packed in a plastic pot, and seeds of cucumber (variety: Sagami Hanjiro) were sowed therein and grown for 12 days in a greenhouse. A PDA medium containing spores of Botrytis cinerea was placed on the surface of leaves of the cucumber to inoculate the surface of leaves of the cucumber. Then, the wettable powder of each of the invented compounds (1) and (2) and the comparative compound (A) was diluted with water to a concentration of 200 ppm and was sprayed onto the foliage of the cucumber so that a sufficient amount of the compound would be applied to the surface of leaves of the cucumber. After spraying, the plant was air-dried and left at 12° C. for 2 days under high humidity, and then the area of lesions was examined.

As a result, the lesion area on the plant treated with the invented compounds (1) and (2) was respectively less than 11% and less than 15% of the lesion area on the plant of untreated plant. The lesion area on the plant treated with the comparative compound (A) was 98% of the untreated plant.

### Test Example 3

Test for showing preventive effect on Sclerotinia rot disease of cucumber (Sclerotinia sclerotiorum)

Sand soil was packed in a plastic pot, and seeds of cucumber (variety: Sagami Hanjiro) were sowed therein and grown for 12 days in a greenhouse. Each of the invented compounds (1)-(5) was formulated into flowable formulation in accordance with Formulation Example 6, and it was diluted with water to a given concentration (500 ppm) and was sprayed onto the foliage of the cucumber so that a sufficient amount of the compound would be applied to the surface of leaves of the cucumber. After spraying, the plant was air-dried and PDA medium containing hyphae of Sclerotinia sclerotiorum was placed on the surface of leaves of the cucumber. After the inoculation, the plant was left at 18° C. for 4 days under high humidity, area of lesions was examined. As a result, the lesion area on the plant treated with the invented compounds (1)-(5) was less than 10% of the lesion area on the untreated plant.

### Test Example 4

Test for showing preventive effect on rice blast disease (Magnaporthe grisea)

Bed soil was packed in a plastic pot, and seeds of rice (variety: Nihonbare) were sowed therein and grown for 12 days in a greenhouse. Each of the invented compounds (1)-(3) and the comparative compound (A) was formulated into flowable formulation in accordance with Formulation Example 6, and it was diluted with water to a given concentration (500 ppm) and was sprayed onto the foliage of the rice so that a sufficient amount of the compound would be applied to the surface of leaves of the rice. After spraying, the plant was air-dried and pots containing leaves infected with blast disease were placed around the sprayed plant. All of the rice were kept under high humidity only in night, and after 5 days from the inoculation, area of lesions was examined. As a result, the lesion area on the plant treated with the invented compounds (1)-(3) was less than 30% of the lesion area on the untreated plant. The lesion area on the plant treated with the comparative compound (A) was 98% of the untreated plant.

1. An amide compound represented by the following formula (I): 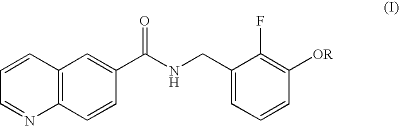
(in the formula, R represents a C1-C5 straight-chain alkyl group).

2. An amide compound according to claim 1, wherein R represents a methyl group or an ethyl group.

3. A plant disease controlling agent containing the amide compound of claim 1 as an active ingredient.

4. A method for controlling plant diseases which includes a step of applying an effective amount of the amide compound of claim 1 to plants or soils.

5. Use of the amide compound of claim 1 for controlling plant diseases by applying the amide compound to plants or soils.

6. An amide compound represented by the following formula (V) or a salt thereof:

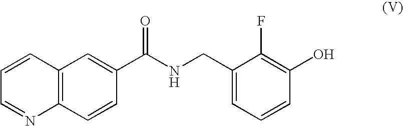

7. An amine compound represented by the following formula (VII) or a salt thereof: [chemical structure]
